(19) United States
(12) Patent Application Publication
(10) Pub. No.: US 2006/0030908 A1
(43) Pub. Date: Feb. 9, 2006

(54) Skin treatment phototherapy device

(75) Inventors: Steven D. Powell, Orem, UT (US); Ryan H. Savage, Salt Lake City, UT (US); Eliot Jacobsen, Orem, UT (US)

Correspondence Address: STOEL RIVES LLP - SLC, 201 SOUTH MAIN STREET, ONE UTAH CENTER, SALT LAKE CITY, UT, 84111, US

(73) Assignee: Lumiport, LLC

(21) Appl. No.: 11/199,971

(22) Filed: Aug. 9, 2005

Related U.S. Application Data
(60) Provisional application No. 60/522,060, filed on Aug. 9, 2004.
(60) Provisional application No. 60/593,152, filed on Dec. 15, 2004.

(51) Int. Cl. A61N 5/06

(52) U.S. Cl. 607/88

(57) ABSTRACT

A light emitting diode (LED) phototherapy device is disclosed. The phototherapy device may be used in the treatment of various skin conditions. The phototherapy device may include multi-color LEDs for emitting multiple wavelengths of light for skin treatment. Furthermore, the phototherapy device may include a control system that receives, from a user, an indication of the skin condition to be treated, and in response the phototherapy device provides the corresponding wavelengths, intensity levels, and time interval for treatment of the skin condition. The phototherapy device may comprise a clamshell structure, pen shape, facial mask, or desk lamp design.

### RELATED APPLICATIONS

This application claims the benefit of U.S. Provisional Patent Application Ser. No. 60/522,060, filed Aug. 9, 2004 and entitled PORTABLE LED DEVICE FOR SKIN CONDITIONS, and U.S. Provisional Patent Application Ser. No. 60/593,152, filed Dec. 15, 2004 and entitled PORTABLE LED LIGHT THERAPY DEVICE FOR SKIN CONDITIONS, which are incorporated herein by reference.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1A is a perspective view of a phototherapy device used in the treatment of skin conditions;

FIG. 1B is a side elevation view of the phototherapy device of FIG. 1A;

FIG. 2 is a side elevation view of another embodiment of a phototherapy device and a recharging base station;

FIG. 3A is a perspective view of another embodiment of a phototherapy device used in the treatment of skin conditions as shown in an open configuration;

FIG. 3B is a perspective view of the phototherapy device of FIG. 3A as shown in a closed configuration;

FIG. 4A is a perspective view of another embodiment of a phototherapy device used in the treatment of skin conditions;

FIG. 4B is an alternative perspective view of the embodiment of the phototherapy device of FIG. 4A;

FIG. 5 is a perspective view of another embodiment of a combination desk lamp device and phototherapy device; and

FIG. 6 is a block diagram of a system for treating various skin conditions with a phototherapy device.

### DETAILED DESCRIPTION

Reference is now made to the figures in which like reference numerals refer to like elements. For clarity, the first digit of a reference numeral indicates the figure number in which the corresponding element is first used. While the various aspects of the embodiments disclosed are presented in drawings, the drawings are not necessarily drawn to scale.

Those skilled in the art will recognize that the systems and methods disclosed can be practiced without one or more of the specific details, or with other methods, components, materials, etc. In some cases, well-known structures, materials, or operations are not shown or described in detail. Furthermore, the described features, structures, or characteristics may be combined in any suitable manner in one or more embodiments. It will also be readily understood that the components of the embodiments as generally described and illustrated in the figures herein could be arranged and designed in a wide variety of different configurations.

For this application, the phrases “connected to” and “coupled to” refer to any form of interaction between two or more entities, including mechanical, electrical, magnetic, electromagnetic, fluid, and thermal interaction. Two components may be coupled to each other even though they are not in direct contact with each other.

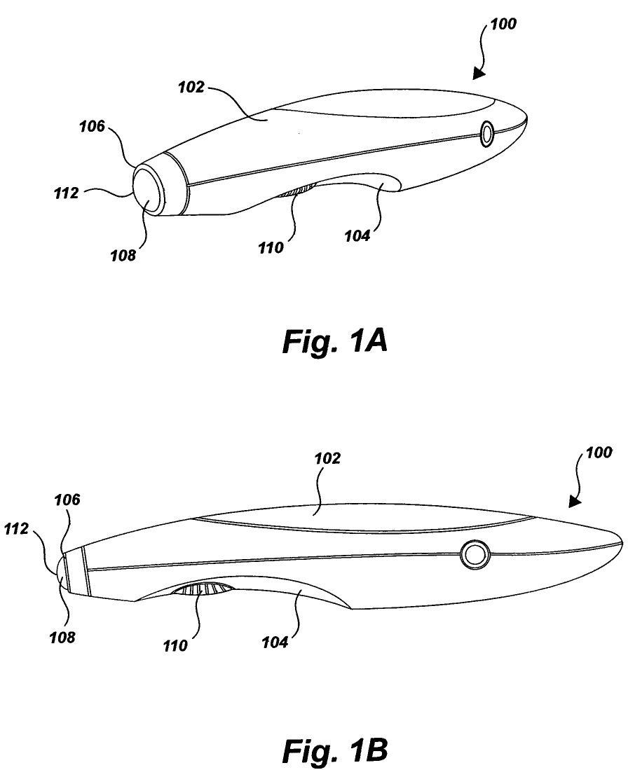

FIG. 1A represents one embodiment of a phototherapy device 100 used in the treatment of various skin conditions, as shown from a perspective view. FIG. 1B represents the phototherapy device 100 as shown from a side elevation view. Referring collectively to FIGS. 1A and 1B, the phototherapy device 100 has a housing 102 that may include a handle 104 in the shape of a handheld pen-like structure. At an output end 106 of the phototherapy device 100, a light emitting diode (“LED”) 108 is located such that light emitting from the LED 108 may be directed substantially collinear with the device's longitudinal axis. In alternative embodiments, more than one LED 108 may be located at the output end 106 of the pen phototherapy device 100.

The LED 108 may be activated when a user depresses a button 110 or switch disposed on the exterior of the housing 102. Once activated, the LED 108 emits light in a narrow range of wavelengths. Since the LED 108 emits a narrow range of wavelengths, often the light emitted is considered monochromatic. LEDs 108 typically use less power, produce less heat, and have a longer life span than most incandescent lamps. Furthermore, LEDs 108 are often an inexpensive alternative to wavelength selection compared to lamp and filter systems. Furthermore, the compactness and portability of an LED phototherapy device 100 are typically superior to alternative lamp and filter designs.

According to one embodiment, the LED 108 is a multi-color LED in a single LED package, which is capable of emitting more than one discrete range of wavelengths. For example, in one embodiment the multi-color LED 108 is a bi-color, or bi-polar LED producing two discrete ranges of wavelengths. The multi-color LED 108 may produce a narrow band of wavelengths in the red portion of the visible electromagnetic spectrum as well as a narrow band of wavelengths in the blue portion of the visible electromagnetic spectrum. The red wavelengths may range between 630 nanometers and 680 nanometers, while the blue wavelengths may range between 400 nanometers and 470 nanometers. In one embodiment, the red band is between 650 to 670 nanometers and the blue band is between 405 to 420 nanometers.

The multi-color LED 108 may be capable of producing just red wavelengths at one time, or just blue wavelengths, or both red and blue wavelengths simultaneously. In other embodiments, the multi-color LED 108 is a tri-color LED producing three discrete ranges of wavelengths. As would be apparent to those having skill in the art, a multi-color LED 108 may be used which can produce more than three discrete wavelengths as the advancement of technology permits.

The LED phototherapy device 100 of FIGS. 1A and 1B may be used to treat a variety of skin conditions. The output end 106 of the device 100 is directed toward or placed on a region of skin having a particular skin condition so that the skin may be treated with LED light therapy. The depicted phototherapy device 100 is small and portable so that small focused light may be directed, for example, around the eyes of a user or other small specific areas where skin conditions may exist that larger light devices may not be able to treat.

The phototherapy device 100 produces specific wavelengths to treat a number of skin conditions. For example, for the treatment of acne both blue wavelengths (400 to 470 nanometers) and red wavelengths (630 to 680 nanometers) may be used. Furthermore, for the treatment of acne, the phototherapy device 100 may provide twice as much exposure to blue wavelengths than to red wavelengths in a single treatment event. Relative exposures of red and blue wavelengths may be determined through a quantifiable value such as light intensity or duration of exposure.

In order to treat wrinkles in the skin, blue, red and yellow wavelength bands may be used. The blue and red wavelength ranges are 400 to 470 nanometers and 630 to 680 nanometers, respectively. The yellow band of wavelengths may be between 530 nanometers and 600 nanometers.

In treating rosacea a yellow range of wavelengths may be used between 530 and 600 nanometers.

In treating sun spots, a yellow range of wavelengths (530 to 600 nanometers) may be used. For alternative forms of sun damage, a red band (630 to 680 nanometers) may be employed.

Blue light (between 400 and 470 nanometers) may be used to treat and kill bacteria that may cause various forms of skin blemishes, such as acne.

Inflammation may be treated by exposing affected skin to red wavelengths (630 to 680 nanometers) and also to infrared wavelengths, which may range from about 800 nanometers to about 1000 nanometers. As discussed above, the two wavelength ranges may be produced by a single multi-color LED 108 or by two separate LEDs, or an array of LEDs as would be apparent to those having skill in the art.

Lesions in the skin may be treated by illuminating the affected area with red wavelengths (630 to 680 nanometers) and infrared wavelengths (800 to 1000 nanometers).

Canker sores may also be treated by irradiating the sore to red and infrared wavelengths (630 to 680 nanometers and 800 to 1000, nanometers, respectively). A typical one time treatment of canker sores may have a duration of exposure between 5 and 15 minutes, with an intensity of approximately 105 mW/cm². However, multiple applications may be necessary in certain situations.

Skin blemishes may be treated through exposure to red, blue and yellow wavelengths. As discussed above the wavelength ranges may be 630 to 680 nanometers for red, 400 to 470 nanometers for blue, and 530 to 600 nanometers for yellow.

LEDs 108 that emit a band of wavelengths in the green portion of the visible electromagnetic spectrum may also be used in treating sun spots, rosacea and wrinkles. The wavelength range associated with green light may range between 500 nanometers and 530 nanometers. LED light therapy may also be used in treating dead skin and other skin problems.

The phototherapy device 100 shown in FIGS. 1A and 1B may also include a lens 112 at its output end 106 to diffuse ultra violet light or other harmful rays that may inadvertently be emitted from the device 100. Furthermore, the LED 108 may be removable from the device 100 and can be replaced with another color LED or another multi-color LED for treatment of a different skin condition.

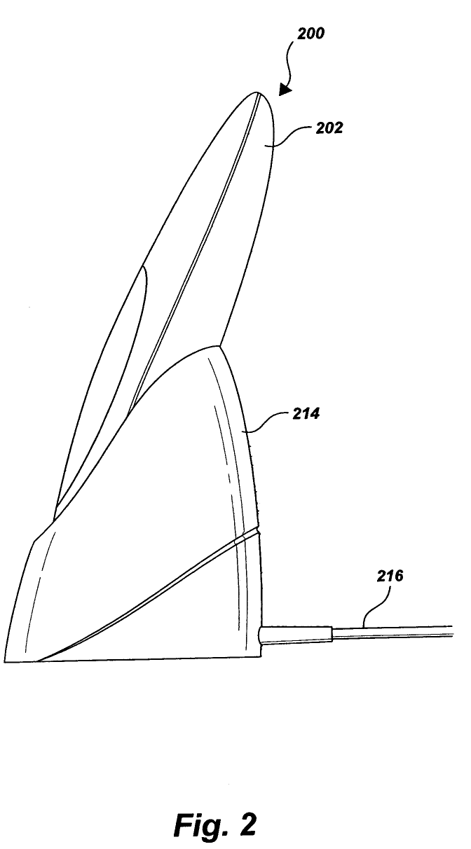

Referring to FIG. 2, another embodiment of a phototherapy device 200 is depicted from a side elevation view. The phototherapy device 200 is similar to the device disclosed in FIGS. 1A and 1B, however the phototherapy device 200 of FIG. 2 comprises a rechargeable power supply, such as a rechargeable battery (not shown). The rechargeable battery may be disposed inside the housing 202 of the device 200.

The phototherapy device 200 is depicted as being cradled in a recharging base station 214. In the cradle position depicted, the base station 214 may have contact points that are in electronic communication with contact points of the phototherapy device 200. The base station 214 is also connected to an AC power supply through a power cord 216. Alternatively, the phototherapy device 200 may be recharged using an AC adapter.

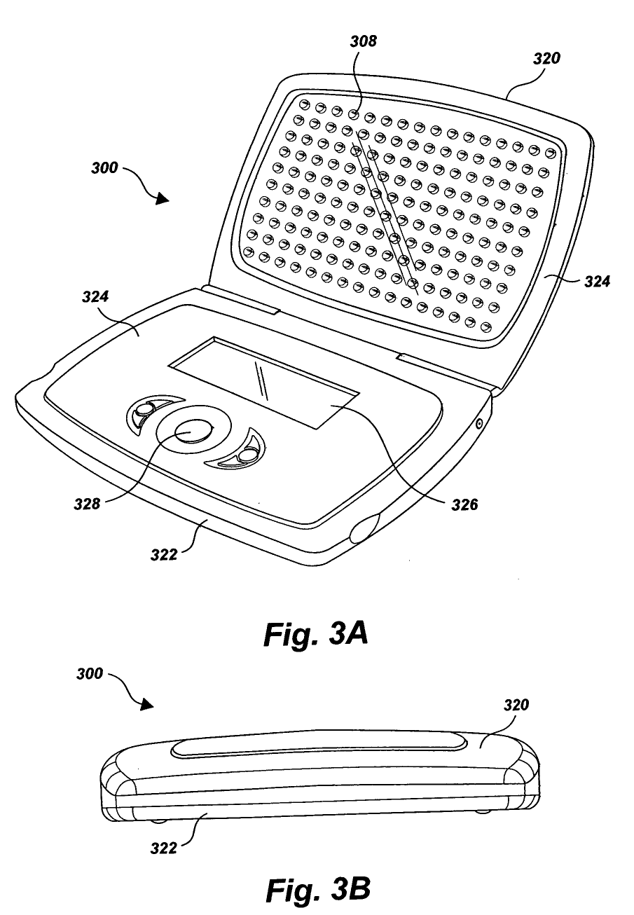

FIGS. 3A and 3B show another embodiment of a phototherapy device 300 used in the treatment of various skin conditions. In FIG. 3A the device 300 is shown in an open configuration from a perspective view. FIG. 3B shows the device 300 in a closed configuration from a perspective view.

The phototherapy device 300 includes a first panel 320 that is hingedly coupled to a second panel 322 in a clamshell-like arrangement. In the open configuration, the internal faces 324 of each panel 320, 322 are exposed to a user, and the first 320 and second 322 panels are arranged at an angle with respect to each other. The angle between panels 320, 322 may be adjustable. In the configuration shown in FIG. 3A, the angle is greater than 90 degrees.

The first 320 and second 322 panels may hingedly move from the open configuration to the close configuration where the panels 320, 322 are located substantially parallel to and adjacent each other. The internal faces 324 are no longer exposed to a user in the closed configuration. According to the embodiment depicted, the first 320 and second 322 panels are similarly sized, in that their internal faces 324 have approximately the same area.

The first panel 320 may include an array of LEDs 308 disposed on its internal face 324. In the open configuration, the array 308 is exposed such that it may be used for treatment of a user's skin. The phototherapy device 300 may optionally include an integrated stand (not shown), so that the device can rest on the stand when in the open configuration, exposing the user to LED light.

In one embodiment, the LED array 308 contains a plurality of red and blue LEDs. In some embodiments, each LED is a single color LED, while in other embodiments, multi-color LEDs may be used. In the single color LED embodiment, the red and blue LEDs may be arranged in a checkerboard configuration, where every other LED emits blue wavelengths while all other adjacent LEDs emit red wavelengths.

Alternatively, other color LEDs may be used, particularly those that are capable of emitting yellow, green and infrared wavelengths. The array of LEDs 308 may also be programmed to emit a combination of wavelengths simultaneously to treat different skin conditions at the same time. Furthermore, the device 300 may also emit different intensities of light. For example, a user may control the intensities of all or some of the LEDs in the LED array 308. The intensities of each color may also be varied independently.

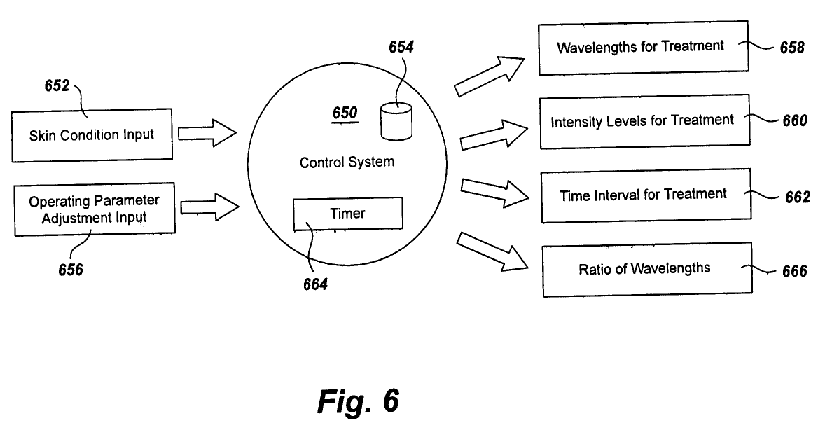

The second panel 322 of the phototherapy device 300 includes a control system for the phototherapy device 300. The functions of the control system will be discussed in greater detail in conjunction with the discussion accompanying FIG. 6. The second panel 322 may include a display 326, such as an LCD display for prompting a user for input or indicating operating status, etc. The second panel 322 may also include mechanical buttons 328 for receiving user input to control the operation of the phototherapy device 300. Alternatively, an LCD touch screen, membrane buttons, or voice activation and recognition may be used to receive user input as would be apparent to those having skill in the art.

The phototherapy device 300 may also be powered by an internal or external portable power source, such as a battery. The battery power source may provide the LED array 308 with power such that AC power is not required. Alternatively, an AC adapter or direct AC connection may be used in other embodiments.

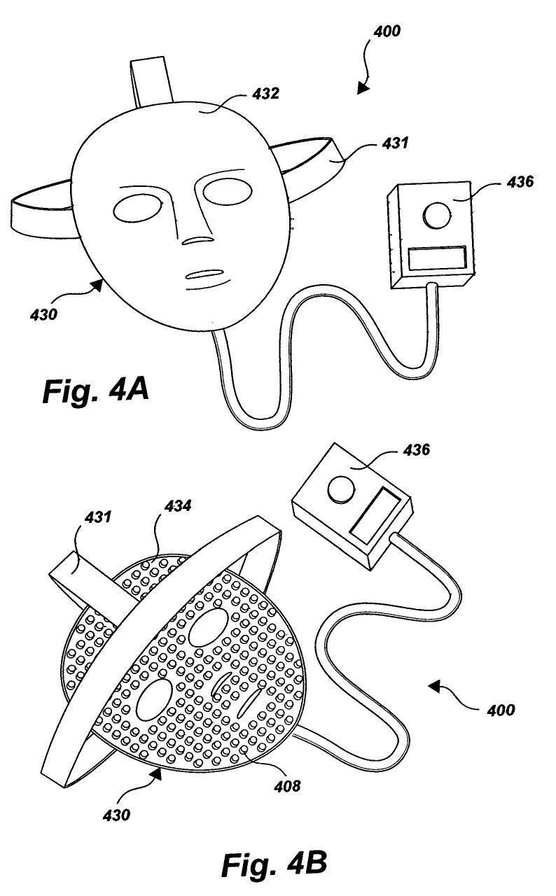

Referring to FIGS. 4A and 4B, an alternative embodiment of a phototherapy device 400 used in the treatment of skin conditions is shown. The device 400 is a facial mask having a mask body 430 that is shaped to cover a substantial portion of a user's face. Covering a substantial portion may consist of covering a user's nose and mouth region, similar to a dust mask, or it could also encompass a larger region encompassing a user's cheeks, chin, nose and mouth, similar to a surgical mask. Alternatively, the facial mask could cover a user's forehead, cheeks and chin. According to the embodiment depicted, the mask body 430 may cover substantially all of a user's face leaving space for a user's eyes and breathing orifices for the nose and/or mouth. A harness 431 or similar device may be used to secure the mask body 430 to a user's face during treatment.

FIG. 4A shows an exterior side 432 of the mask body 430. FIG. 4B shows an interior side 434 of the mask body 430. The facial mask device 400 includes an LED array 408 that is embedded in the interior side 434 of the mask body 430, so that the LEDs 408 are positioned to emit light directly toward a user's skin when wearing the device 400. In one embodiment, the LED array 408 may include red, yellow and blue LEDs scattered throughout the interior portion of the mask body 430 to treat wrinkles. Alternative LED arrangements and LED types may be incorporated into the facial mask phototherapy device 400 as would be apparent to those having skill in the art, such as including green and infrared LEDs and other color combinations of LEDs.

The device 400 may further include a controller 436 in electronic communication with the mask body 430 and LED array 408. The controller 436 may allow the user to select specific red, yellow or blue wavelengths, or a combination thereof to treat various skin conditions. Additional LED color types may also be used. Alternatively, the controller 436 may be as simple as a device for switching on and off the LED array 408. The controller 436 may optionally include a display that assists a user in selecting and controlling treatment modes, timers, and other functionality features. For example, treatment modes may include activation of blue LEDs, activation of red LEDs, activation of yellow LEDs, activation of all three colors, or any other combination thereof. The controller 436 may also include a portable power supply to increase the portability of the device 400.

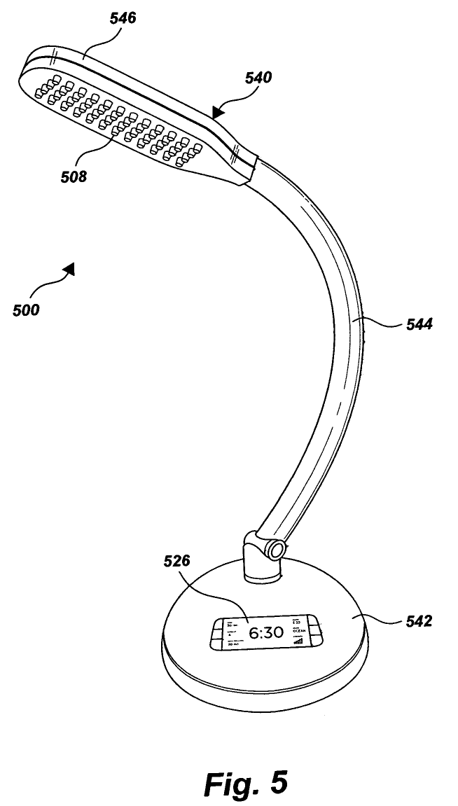

FIG. 5 represents another embodiment of a phototherapy device 500 that is integrated with a desk lamp device 540, as shown from a perspective view. The desk lamp 540 may include a base 542 and a lamp neck 544 and lamp head 546. The desk lamp 540 may also include a display 526, such as an LCD display for prompting a user for input or indicating operating status, etc., similar to the display described in conjunction with FIG. 3A.

Embedded in the lamp head 546 is an LED illumination source 508, such as an array of LEDs. The desk lamp 540 may produce white light for general lighting purposes from the LED array 508, or from a different white light source, such as an incandescent lamp or a fluorescent lamp. The desk lamp 540 may also produce wavelength specific light from the LED illumination source 508 for the treatment of various skin conditions. Alternatively, the desk lamp 540 may provide both white light and wavelength-specific light, simultaneously. The LED array 508 may comprise a plurality of multi-colored LEDs. As with the phototherapy devices heretofore described, the phototherapy device 500 of FIG. 5 may have the capabilities of changing wavelengths to treat various skin conditions as selected by the user.

Alternative devices, other than those heretofore disclosed, may also be used in accordance with the LED light therapy principles described. For instance, multi-color LEDs or multiple color LED therapy programs may be incorporated into a device that is large enough to provide LED exposure to most of a user's body. A user may stand in front of such a device, or alternatively, lie down in a device similar to a tanning bed. Such a device may include a large array of LEDs.

Furthermore, LEDs, such as multi-color LEDs may be embedded into a fabric swath or belt allowing a user to wrap the belt around a specific area of the user's body for treatment of a particular region of skin. For example, an LED fabric belt may include infrared LEDs, or other colored LEDs to treat chronic or other forms of pain, swelling, inflammation, etc. The fabric device may be wrapped around the affected region of skin to assist in the reduction of swelling, increasing blood flow, or aiding in the body's process of tissue repair. The LED fabric belt may be in electronic communication with a controller and portable power device. The controller would allow a user to select operation parameters such as time intervals, intensities, and wavelength options.

FIG. 6 is a block diagram of a control system 650 for treating various skin conditions with an LED phototherapy device. The control system 650 may be incorporated, in part, into a device controller as heretofore described. The control system 650 may receive various forms of user input in order to control various treatment modes of the phototherapy device.

For example, a user may provide input 652 indicative of a skin condition that a user desires to be treated by the LED phototherapy device. Examples of various skin condition inputs 652 may include acne, rosacea, wrinkles, inflammation, sun spots or sun damage, bacteria, blemishes, lesions or canker sores. A user may select one or more of a list of skin conditions to be treated and the control system 650 accesses operating parameters stored on a memory device 654 or database in machine readable form. The operating parameters of the phototherapy device that correspond with a particular light therapy treatment may be inputted by a manufacturer or programmer of the device, or alternatively a user may provide adjustment operating parameter input 656 in accordance with a customized LED skin treatment program.

The control system 650 accesses the memory device 654 containing multiple operating parameters and selects those corresponding to the skin condition input 652 received. The phototherapy device then runs according to the operating parameters corresponding with the selected skin condition input 652. One example of an operating parameter output of the control system 650 is a control signal corresponding to the specific wavelengths for treatment 658 of the skin condition selected. Accordingly, if acne is selected by the user, the control system 650 accesses the corresponding operating parameter that indicates both red and blue wavelengths are to be used for treatment. However, if the user selected rosacea as the skin condition to be treated, the wavelengths for treatment 658 may be in the yellow band (530 to 600 nanometers).

Another form of output of the control system 650 is the operating parameter that indicates the intensity levels 660 for treatment of the skin condition selected. For example, with the phototherapy device disclosed in FIGS. 1A and 1B, the intensity levels of a multi-color LED may be 105 mW/cm². However, with the phototherapy device disclosed in FIGS. 3A and 3B, an intensity level output 660 of 92 mW/cm² may be provided by the control system 650. A user may adjust the intensity level output 660 corresponding to a particular skin treatment. The user adjusts that particular operating parameter through input 656 indicating an increase or a decrease in intensity to treat more severe or less severe skin conditions, respectively. Intensity adjustments may be made, for example, in percentage increments such as ±5%, ±10%, ±15%, etc.

Another operating parameter that may be controlled is the time interval for treatment 662. A typical treatment session may last 15 minutes for most skin conditions. However, treatment for canker sores may be less, such as between 5 and 15 minutes, depending upon the user input. Furthermore, certain treatments using the pen device may last for 3 minutes as desired by the user. The time interval for treatment 662 may be controlled by a timer 664, which may be embodied, for example, as a Real Time Clock (RTC). Once the skin condition input 652 is received and the corresponding operating parameters accessed, the indicated time interval 662 is controlled by the timer 664. Once the timer 664 reaches the time interval 662 indicated it automatically shuts off LED emission of the phototherapy device.

Additionally, the operating parameters corresponding to a skin condition input 652 may include wavelength ratio data 666. For example, when acne is selected as the skin condition to be treated, the operating parameters corresponding with the treatment of acne would indicate that twice as much exposure to blue wavelengths as compared to red wavelengths is desired. Consequently, the wavelength ratio 666 for acne would be 2:1, blue to red. The relative exposures of red and blue wavelengths may be determined through a quantifiable value such as light intensity or duration of exposure. Therefore, blue LED light may be emitted at twice the intensity of red LED light. Alternatively, the exposure time of blue LED light during a particular treatment interval would be twice as long as red LED light. This may be accomplished by pulsating blue LEDs twice as much as red LEDs, or by activating twice as many blue LEDs than red LEDs, or other methods known to those having skill in the art.

Accordingly, a user is able to control the wavelengths emitted, the intensity levels, the time intervals for treatment, and the relative ratio of wavelengths produced by simply selecting a particular skin condition. By selecting the skin condition, the control system 650 causes the LED phototherapy device to provide the appropriate colors, intensity, etc., for that skin condition.

The control system may be in electronic communication with a display, such as an LCD display discussed in conjunction with the description of FIG. 3A. By way of example, the LCD display may show an indication of the skin condition selected by the user and the associated operating parameters. In some embodiments, the display may show a countdown of time left or time elapsed for the particular light therapy treatment. Furthermore, an audible alert, such as a beep, may let the user know when the treatment event has ended.

While specific embodiments and applications of phototherapy devices have been illustrated and described, it is to be understood that the invention claimed hereinafter is not limited to the precise configuration and components disclosed. Various modifications, changes, and variations apparent to those of skill in the art may be made in the arrangement, operation, and details of the devices and systems disclosed.

What is claimed is:

1. A phototherapy device, comprising:
at least one multi-color light emitting diode (LED) capable of emitting more than one discrete range of wavelengths of light; and
a housing that contains the LED, such that light emitting from the LED may be directed onto a region of a user's skin;
wherein the ranges of wavelengths are selected to treat a skin condition present on the region of skin.

2. The phototherapy device of claim 1, wherein the multi-color LED is a bi-color LED.

3. The phototherapy device of claim 2, wherein the bi-color LED is bi-polar and emits a range of wavelengths in a blue portion of the visible electromagnetic spectrum and a range of wavelengths in a red portion of the visible electromagnetic spectrum.

4. The phototherapy device of claim 3, wherein the range of blue wavelengths is between 400 nanometers and 470 nanometers and the range of red wavelengths is between 630 nanometers and 680 nanometers.

5. The phototherapy device of claim 1, wherein the multi-color LED is capable of emitting more than two discrete ranges of wavelengths of light.

6. The phototherapy device of claim 1, wherein the skin condition is at least one of: acne, rosacea, wrinkles, inflammation, sun damage, bacteria, blemishes and lesions.

7. The phototherapy device of claim 6, further comprising a control system to control the LED according to operating parameters, the operating parameters including at least one of intensity level of LED emission, duration of LED emission, and wavelength selection, such that the user selects the skin condition to be treated whereupon the control system controls the LED in accordance with the operating parameters corresponding to treatment of the selected skin condition.

8. The phototherapy device of claim 1, further comprising a plurality of LEDs arranged in an array.

9. The phototherapy device of claim 1, wherein the device is a portable device further comprising a rechargeable battery.

10. The phototherapy device of claim 9, wherein the housing includes a handle for the user to grasp and an output end for directing emission of the LED.

11. The phototherapy device of claim 9, wherein the housing is shaped as a facial mask having a mask body that is adapted to cover a substantial portion of the user's face, such that an array of LEDs are positioned to emit light toward the user's skin when wearing the mask.

12. The phototherapy device of claim 1, wherein the housing comprises two panels hingedly coupled such that the housing has a closed configuration and an open configuration, the open configuration exposing an array of LEDs disposed on at least one of the panels.

13. The phototherapy device of claim 1, wherein the housing is integrated into a desk lamp device, such that, upon user selection, the desk lamp provides white light or LED light to treat the skin condition or a combination of white and LED light.

14. A phototherapy device for the treatment of skin conditions, comprising:
a light emitting diode (LED) illumination source capable of producing at least one range of wavelengths of light to be directed onto a user's skin;
a machine readable medium for storing operating parameters of the LED illumination source, the operating parameters corresponding to treatment of skin conditions; and
a control system to receive input from the user indicative of a skin condition to be treated, such that the control system accesses the operating parameters corresponding to the indicated skin condition and the control system controls the LED illumination source in accordance with the corresponding operating parameters;
wherein the operating parameters include at least one range of wavelengths for treatment of each skin condition.

15. The phototherapy device of claim 14, wherein the operating parameters further include at least one intensity level of the light produced by the LED illumination source for treatment of each skin condition.

16. The phototherapy device of claim 14, wherein the operating parameters further include at least one time interval representing a length of time the LED illumination source emits light for treatment of each skin condition.

17. The phototherapy device of claim 16, wherein the control system comprises a timer which is set according to the at least one time interval of the operating parameters corresponding to the indicated skin condition, such that emission of the LED illumination source is automatically discontinued when the at least one time interval has elapsed.

18. The phototherapy device of claim 14, wherein the operating parameters further include at least one wavelength range ratio representing how much of a quantifiable value of one range of wavelengths is emitted relative to the quantifiable value of another range of wavelengths.

19. The phototherapy device of claim 14, wherein the operating parameters are adjustable by the user.

20. The phototherapy device of claim 14, wherein the LED illumination source comprises an array of LEDs.

21. The phototherapy device of claim 20, wherein the array of LEDs are arranged in a housing, the housing comprising two panels hingedly coupled to each other such that the housing has a closed configuration and an open configuration, where the array of LEDs is disposed on at least one of the panels and are exposed when the housing is in the open configuration.

22. The phototherapy device of claim 14, wherein the LED illumination source comprises at least one multi-color LED.

23. The phototherapy device of claim 14, wherein the skin condition to be treated is at least one of: acne, rosacea, wrinkles, inflammation, sun damage, bacteria, blemishes and lesions.

24. The phototherapy device of claim 14, further comprising a display in electronic communication with the control system, the display showing the skin condition inputted by the user.

25. The phototherapy device of claim 14, further comprising a display in electronic communication with the control system, the display showing a timer indicating a treatment time.

26. A phototherapy device, comprising:
a light emitting diode (LED) illumination source producing at least one range of wavelengths of light, the range of wavelengths is to treat oral lesions; and
a housing that contains the LED illumination source to direct light emitting from the LED illumination source onto an oral lesion;
wherein a portion of the housing is sized to be inserted into a user's mouth.

27. The phototherapy device of claim 26, wherein the at least one range of wavelengths is in a red portion of the visible electromagnetic spectrum.

28. The phototherapy device of claim 26, wherein the at least one range of wavelengths is in an infrared portion of the electromagnetic spectrum.

29. The phototherapy device of claim 26, wherein the oral lesion is a canker sore.

30. The phototherapy device of claim 29, wherein the LED illumination source produces more than one range of wavelengths of light, such that the ranges of wavelengths comprise a first range of wavelengths in a red portion of the visible electromagnetic spectrum and a second range of wavelengths in the infrared portion of the electromagnet spectrum.

31. The phototherapy device of claim 30, wherein the first range of wavelengths is between 630 nanometers and 680 nanometers and the second range of wavelengths is between 800 nanometers and 1000 nanometers.

32. The phototherapy device of claim 26, wherein the range of wavelengths is to treat at least one of: oral blisters, oral inflammation, and oral infections.

33. A phototherapy device for the treatment of skin conditions, comprising:
a facial mask having a mask body that is shaped to cover a substantial portion of a user's face;
an array of light emitting diodes (LEDs) embedded into the mask, such that the LEDs are positioned to emit light toward a user's skin when wearing the mask; and
a controller in electronic communication with the facial mask, the controller for controlling the array of LEDs.

34. The phototherapy device of claim 33, wherein the array of LEDs emit more than one range of wavelengths of light to treat different skin conditions.

35. The phototherapy device of claim 34, wherein the controller is to control treatment modes as selected by the user, the treatment modes selected from the group consisting of activation of the LEDs producing a first range of wavelengths, activation of the LEDs producing a second range of wavelengths, and activation of the LEDs producing both first and second ranges of wavelengths.

36. The phototherapy device of claim 35, further comprising a machine readable medium for storing operating parameters of the array of LEDs, the operating parameters corresponding to treatment modes selected by the user.

37. The phototherapy device of claim 36, wherein the operating parameters include at least one of: at least one range of wavelengths for treatment of each skin condition, at least one intensity level of light produced by the array of LEDs for treatment of each skin condition, and at least one time interval representing a length of time the array of LEDs emits light for treatment of each skin condition.

38. The phototherapy device of claim 33, further comprising a portable power supply in electronic communication with the array of LEDs and the controller.

39. The phototherapy device of claim 33, wherein the mask body is shaped to cover at least the user's nose and mouth.

40. The phototherapy device of claim 39, wherein the mask body is shaped to cover at least the user's cheeks and chin.

41. The phototherapy device of claim 33, wherein the mask body is shaped to cover substantially all of the user's face.
